(19) United States
(12) Patent Application Publication
(10) Pub. No.: US 2006/0246056 A1
(43) Pub. Date: Nov. 2, 2006

(54) Immunotherapy of rectal cancer

(75) Inventors: Hans Loibner, Vienna (AT); Helmut Eckert, Obervil (CH); Gottfried Himmler, Vienna (AT)

Correspondence Address: BIRCH STEWART KOLASCH & BIRCH, PO BOX 747, FALLS CHURCH, VA, 22040-0747, US

(73) Assignee: IGENEON KREBS-IMMUNTHERAPIE FORSCHUNGS-UND ENTWICKLUNGS-AG, VIENNA (AT)

(21) Appl. No.: 10/558,166

(22) PCT Filed: May 24, 2004
(86) PCT No.: PCT/EP04/05541

§ 371 Date: Nov. 23, 2005

(30) Foreign Application Priority Data
May 27, 2003 (AT) A 821/2003

(51) Int. Cl. A61K 39/395

(52) U.S. Cl. 424/131.1; 424/155.1

(57) ABSTRACT

Described is the use of an immunotherapeutic agent targeting a human tumor associated antigen for the production of a medicament for immunotherapy of patients with rectal cancer or being at risk of rectal cancer, wherein the immunotherapeutic agent is a vaccine, an antibody or derivative or fragment thereof.

The present invention relates to the use of a immunotherapeutic agent of a human tumor associated antigen for immunotherapy of patients at risk of rectal cancer. The invention further refers to a pharmaceutical preparation comprising the immunotherapeutic agent and a method of application of this preparation.

In industrialized countries, cancer is the second leading cause of death. The yearly incidence of new cancer cases worldwide is estimated to be 7 millions. The most prominent indications (ap- prox. 70%) are cancers of epithelial origin - including breast, colorectal, gastric, pancreatic, lung, prostate, ovarian (Black et al., 1997, Eur J Cancer, 33, 1075-107). Up to date, surgery, chemotherapy and radiation therapy are the generally accepted standards. Despite some progress in treatment of certain tumor indications and stages, in general the presently available can- cer therapies are not satisfactory, and in particular there is a lack of effective therapies to prevent the detrimental develop- ment of metastases:

In many patients with epithelial cancer, the clinically detectable tumor mass is removed successfully by surgery (surgery with curative intent). For example, the treatment of rectal cancer is frequently associated with colostomy, the primary modality is radical surgical resection. The results of these primarily surgical approaches can be improved with adjuvant therapy, for example chemotherapy or radiation therapy. Although the rectum frequently is considered to be extraperitoneal, the anterior surface of the upper third of the rectum is covered with serosa and is therefore intraperitoneal.

At the time of diagnosis and surgery of a primary tumor, occult single tumor cells frequently have disseminated into various organs of the patient (Cote et al., 1995, Ann Surg, 222, 415-23; discussion 423-5.; Cote et al., 1991, J Clin Oncol, 9, 1749-56). Detection of disseminated tumor cells in lymph nodes, bone marrow and peripheral blood is associated with worse prognosis (Pantel & Otte, 2001, Recent Results Cancer Res, 158, 14-24). These disseminated tumor cells are known to be the cause for the later growth of metastases, often years after diaanosis and surgical removal of all clinically proven tumor tissue. So far, in almost all cases metastatic epithelial cancer is incurable and thus detrimental. In consequence, the overall 5-year survival rate in cancer of epithelial origin is only approx. 50% (Landis et al., 1999, CA Cancer J Clin, 49, 8-31, 1).

Therefore a more effective treatment of “minimal residual cancer”, e.g. destruction of occult single or even micrometastatic cells in order to prevent the growth of macrometastases is an urgent and mostly unmet medical need. For this purpose, conventional chemotherapeutic approaches are rather unsuccessful since micrometastatic cells often are dormant and thus are not an appropriate target for chemotherapies that are effective only in case of rapidly dividing cells (Riethmuller & Klein, 2001, Semin Cancer Biol, 11, 307-11).

Disseminating tumor cells also play a role in later stages of the disease when macrometastases already are present. These cells contribute to further disease spreading, i.e. development of additional metastases (Cavallaro & Christofori, 2001, Biochim Biophys Acta, 1552, 39-45).

There is increasing evidence that disseminated tumor cells are appropriate targets for immunotherapies of cancer. Eg it was shown that antibodies exert therapeutic effects against these tumor cells:

Passive immunotherapy of cancer patients with the murine anti-Lewis Y antibody ABL364 led to substantial reduction of micro-metastatic cells in bone marrow (Schlimok et al., 1995, Eur. J. Cancer, 31A, 1799-1803). Thereby it was first time demonstrated that appropriate antibodies may affect and destroy disseminated tumor cells. A similar observation was made using the murine monoclonal antibody 17-1A as therapeutic agent (Braun et al., 1999, Clin.Cancer Res., 5, 3999-4004)

Patients with resected Dukes C colon cancer (patients after successful surgical removal of primary tumor, but with the risk for already disseminated occult tumor cells) have been treated with the murine monoclonal antibody 17-1A in a controlled adjuvant trial (control group observation). This passive immunotherapy led to a significantly decreased relapse rate and prolonged survival (Riethmuller et al., 1998, J Clin Oncol, 16, 1788-94).

Vaccination of metastatic colorectal cancer patients with a polyclonal goat anti-idiotype antibody preparation (SCV 106) in a placebo controlled trial apparently had no major effect on solid metastases (no partial or complete remissions), but led to significantly increased survival time and reduced further disease spreading in immunologically responding patients (Samonigg et al., 1999, J Immunother, 22, 481-488). Thereby it was shown that specific antibodies induced by vaccination might exert beneficial effects in metastasized cancer patients, probably via destruction of disseminating tumor cells.

The Epithelial Cell Adhesion Molecule (EpCAM), a 40 kDa membrane glycoprotein has been described as a tumor associated antigen by various names originating from the name of the respective monoclonal antibody that was raised against the molecule (e.g. 17-1A, KSA, GA73-3, AUA1 (Durbin et al., 1990, Int. J. Cancer, 45, 562-565; Herlyn et al., 1979; Herlyn et al., 1986, Hybridoma, 5, 3-10.; Ross et al., 1986, Biochem Biophys Res Commun, 135, 297-303). The corresponding cDNA was independently cloned by several groups (Perez & Walker, 1989, J Immunol, 142, 3662-7; Strnad et al., 1989, Cancer Res., 49, 314-31, Szala et al., 1990, Proc.N-atl. Acad.Sci., 84, 214-218).

In humans, this glycoprotein is over-expressed on the surface of almost all cancer cells of epithelial origin (Balzar et al., 1999b, J Mol Med, 77, 699-712) as well as on small cell lung cancer (DeLeu et al., 1994, Int.J.Cancer, 60-63). EpCAM is also detected on cell membranes of all simple, pseudo-stratified and transitional epithelia and thus can be considered as pan-car-cinoma/pan-epithelial marker (Went P. et al., Hum Pathol 2004 Jan 35:122-8).

EpCAM mediates Ca²⁺-independent homotypic cell-cell adhesions (Litvinov et al., 1994, J Cell Biol, 125, 437-46). The formation of EpCAM mediated adhesions has a negative regulatory effect on adhesions mediated by cadherins, which may have strong effects on the growth and differentiation of epithelial cells (Litvinov et al., 1997, J Cell Biol, 139, 1337-48). EpCAM also seems to be involved in the delivery of cellular growth/developmental signals and may have an important role during embryonic development (Cirulli et al., 1998, J Cell Biol, 140, 1519-1534). Details of the molecular and structural biology of EpCAM have been reported (Balzar et al., 2001, Mol Cell Biol, 21, 2570-80), however, the exact role of this molecule in epithelial cell activities remains to be further investigated.

Since EpCAM is strongly expressed at the cell surface of most carcinomas, the molecule is an attractive target for immunological approaches to treat cancer. First clinical trials with antibodies against EpCAM already started in the early 1980s (Herlyn et al., 1991, Am J Clin Oncol, 14, 371-8). After many years of clinical research with a variety of immunotherapeutic approaches and after many rather disappointing results, finally the clinical relevance and practicability of EpCAM as target for both passive and active cancer immunotherapy was proven:
The murine monoclonal antibody 17-1A (clinical efficacy demonstrated for adjuvant passive immunotherapy of resected Dukes C colon cancer patients (Riethmuller et al., 1998, J Clin Oncol, 16, 1788-94) is directed against EpCAM (Gottlinger et al., 1986b, Hybridoma, 5, 29-37).
The polyclonal goat anti-idiotype antibody vaccine SCV 106 (prolongation of survival shown after vaccination of metastatic colorectal cancer patients (Samonigg et al., 1999, J Immunother, 22, 481-488) is designed to mimic EpCAM (Herlyn et al., 1987, Eur J Immunol, 17, 1649-52), vaccination increases number of EpCAM-specific B-cells (Loibner et al., 1990, Lancet, 335, 171).

Increasing the anti-EpCAM reactivity in patients by active or passive therapeutic approaches did not result in any systemic side effects or even autoimmunity (Staib et al. Int J Cancer, 2001, 92, 79-87; Gruber et al., 2000, Cancer Res,. 60, 1921-1926)

Besides EpCAM there are further known tumor associated antigens (TAA), such as the Lewis antigens. Those antigens are overexpressed on various epithelial cancers. Among them there are Lewis y, Lewis x and Lewis b-structures, but also sialylated Lewis x carbohydrates. Further carbohydrate antigens are Globo H structures, KH1, Tn-antigen, sialylTn, TF antigen and alpha-1,3-galactosyl epitope (Electrophoresis, 1999, 20:362; Curr Pharmaceutical Design, 2000, 6:485, Neoplasma, 1996, 43:285). In Durrant et al. (Cancer Research, 1994, 54:4837-4840) it was shown that an anti-idiotypic monoclonal antibody (105AD7) induces antitumor cellular responses in animals and appeared to prolong survival in patients with colorectal cancer without associated toxicity. A prolongation of survival in rectal cancer patients could not be shown.

Other TAA are proteins that are highly expressed on tumor cells, for example CEA, N-CAM, TAG-72, MUC, Folate Binding Protein A-33, CA125, HER-2/neu, EGF-receptors, PSA, MART etc. (Sem.Cancer Bio., 1995, 6:321). Relevant TAA are often surface antigens of epithelial cells that occur on growing cells like fetal tissue or tumor cells. A special group of TAA are involved in cellular adhesion processes of epithelial cells. Among the cellular adhesion proteins, overexpressed on tumor cells, are EpCAM, NCAM and CEA.

The rectum in the adult is approximately 15 cm long. Actual length and division into surgical segments reflect several patient features, such as height, body habitus, pelvic width (gynecoid or android), and curve of the sacral hollow, within which the rectum resides. Frequently the rectum is considered extraperitoneal, the anterior surface of the upper third of the rectum is covered with serosa and is therefore intraperitoneal. Treatment of rectal cancer is usually done by radical surgery and radiation therapy, whereas colon cancer is usually treated by surgery and chemotherapy.

Patients being at risk of rectal cancer have already developed tumors within the rectum, either as primary tumors or metastases, or show predisposition for rectal cancer. The risk for rectal cancer might increase due to genetic disposition. On the other hand patients at risk of rectal cancer may have already developed tumors or metastases in other organs such as the colon, yet spreading of disease might occur then in the rectum.

Patients being at risk of rectal cancer can be patients having a risk of relapse of rectal cancer, which might occur after conventional treatment of the disease.

Clinical behaviour of rectal cancer is different from colorectal cancer: In rectal cancer the major problem is local recurrence whereas in colon cancer it is distant metastasis. This might have a molecular basis eg. P53 mutations and overexpression is a prognostic factor for survival in rectal carcinoma and not in colon carcinoma (J. Pathol. 2001, v195 p171-178). Loss of EPCAM expression of rectal tumors seems to be predictor for local tumor recurrence.

Further differences between colon versus rectum cancer:

Distal tumors (rectal) display a higher frequency of
K-ras mutations (Scott et al., 1993, Gut 34 :621-624)
18q allelic loss (Kern et al., 1989, JAMA, 261:3099-3103)
p53 accumulation (Soong et al., 1997, Clin.Cancer Res., 3, 1405-1411)
c-myc expression (Rothberg et al., 1985, Br.J.Cancer, 52, 629-632)
aneuploidy (Lanza et al., 1996, Am.J.Clin.Pathol., 105, 604-612)
beta-catenin expression (Kapiteijn et al., 2001, J.Pathol., 195, 171-178)
re-expression of blood group determinants (Caldero et al., 1989, Virch.Arch.A. Pathol.Anat.Histopath., 415:3479)
cyclin Dl overexpression (Distler et al., 1997, Dig Dis, 15:302
local recurrence
surgeon has a significant higher influence on survival
less MSIs
higher mucin sulphate content
re-expression of blood group determinants Right-sided (colon) tumors are more often
mucinous (Hanski et al., 1996, Cancer Lett., 103, 163-170)
diploid (Lanza et al., 1996, Am.J.Clin.Pathol., 105, 604-612)
MSI-phenotype (Thibodeau et al., 1993, Science, 260, 816-819)

Based on the progression of the disease rectal cancer is classified in four stages according to the state of the art. Stages III and IV are characterized in that metastases already occur in the lymph nodes, in stage IV metastases are also found in other organs throughout the body.

Conventional treatment of rectal cancer like surgery and treatment with radiation and/or chemotherapy might not be efficient in treating and preventing metastases formation, thus prolonging the survival time. It is therefore an object of the present invention to provide a novel method for the treatment of patients having rectal cancer or being at risk of rectal cancer.

This problem is solved by the method and pharmaceutical preparation as described in the claims. According to the invention there is provided a novel method for the treatment of patients with rectal cancer or being at risk of rectal cancer by immunotherapy with immunotherapeutic agents targeting human tumor associated antigens. Furthermore a pharmaceutical preparation is provided for the treatment of mammals with a risk of rectal cancer.

It was found that immunotherapy of colorectal cancer with an immunotherapeutic agent targeting a tumor associated antigen did in fact lead to increased survival rates in patients. The patients were undergoing active immunotherapy to provoke an immune response that increased the titer of the immunotherapeutic agent in the patients serum. When rectal cancer patients or patients with risk of rectal cancer developed an immune response against the immunotherapeutic agent, surprisingly the survival rate was even more increased than in the colon cancer patients.

Patients being at risk of rectal cancer have already developed tumors within the rectum, either as primary tumors or metastases, or show predisposition for rectal cancer. In these cases the risk for rectal cancer might increase due to genetic disposition.

Patients being at risk of rectal cancer can be patients having a risk of relapse of rectal cancer, which might occur after conventional treatment of the disease. Patients might already have developed metastases, but growth of these metastases can be prohibited or at least reduced by the use of the immunotherapeutic agents according to the invention. As a result, life expectancy and quality of life can be increased.

Furthermore, the treatment according to the invention can be highly effective for treatment of patients having rectal cancer stage III and/or IV.

The immunotherapeutic agents according to the invention can be antibodies or antibody derivatives or fragments thereof. Among the antibody fragments are functional equivalents or homologues of antibodies including any polypeptide comprising an immunoglobulin binding domain or peptides mimicking this binding domain. Chimeric molecules comprising an immunoglobulin binding domain, or equivalents, fused to another polypeptide are therefore included. Preferably, the antibody derivative comprises at least parts of the Fab fragment, preferably together with at least parts of the F(ab′)₂ fragment and/or parts of the hinge region and/or the Fc part of a lambda or kappa antibody. Exemplary antibody molecules are intact immunoglobulin molecules and those portions of an immunoglobulin molecule that contains the paratope, including those portions known as Fab, Fab′, F(ab′)₂and F (v). Preferably, the antibody is an IgG, IgM or IgA antibody.

The antibody or antibody derivative used according to the invention can also be a glycosylated antibody, wherein the glycosylation can also mimick an epitope of a carbohydrate epitope of a tumor associated antigen (TAA).

The antibody or antibody derivative can be of human or animal origin, preferably of mammalian origin, for example of mouse, rat, goat origin. It can be produced by hybridoma technology according to methods well known from the art or by recombinant expression using appropriate expression systems. Depending on the host system used, the antibody or antibody derivative can show specific glycosylation patterns.

The immunotherapeutic agent according to the invention can be an anti-idiotypic antibody, i.e. an ab2 and/or an idiotypic antibody having specificity for a tumor associated antigen, i.e. an ab1.

The immunotherapeutic agent according to the invention can also be a vaccine. This can be an antigenic structure, for example a TAA protein or polypeptide of a TAA which can either alone or together with a vaccine adjuvant induce an immune response against the antigen. The TAA antigen can be either isolated or recombinantly produced by known techniques.

According to the invention the immunotherapeutic agent is preferably employed by active immunization thus inducing a relevant titer against the immunotherapeutic agent in the patient's blood. For immunization purposes either the TAA or a mimic of the TAA, such as anti-idiotypic or mimotopic antibodies, antibody derivatives or other TAA mimicking structures, such as peptides, can be used as immunogenic substance. The term “immunogenic” defines any structure that leads to an immune response in a specific host system. For example, a murine antibody or a fragment thereof is highly immunogenic in humans, even more when combined with adjuvants. The immunogenic substance may provoke an immune response against the respective antibody idiotype or other TAA relevant structures. The immunogenic substance can preferably induce immunogenicity when being denatured or when conjugated to appropriate structures or carriers.

Preferred immunogenic antibodies used according to the invention are for example described in EP 1 140 168, EP 1 230 932, EP 0 644 947 and EP 0 528 767. A preferred antibody used for active immunotherapy is an anti-EpCAM antibody as described in WO 00/41722 or A599/2003.

Preferred tumor associated antigens that are targeted by the immunotherapeutic agent according to the invention are those typically expressed on malignant cells of solid tumors, e.g. TAG-72, MUC1, Folate Binding Protein A-33, CA125, HER-2/neu, EGF-receptors, PSA, MART etc. Suitable antigens are usually expressed in at least 20% of the cases of a particular disease or cancer, preferably in at least 30%, more preferably in at least 40%, most preferably in at least 50% of the cases.

According to the invention preferred relevant TAA are derived from tumor associated aberrant carbohydrate structures, such as Lewis antigens, e.g. Lewis x-, Lewis b- und Lewis y-structures, also sialylated Lewis x-structures, GloboH-structures, KH1, Tn-antigen or sialylTn, TF-antigen and alpha-1-3-galactosyl-epi-tope. According to the invention, an even more preferred TAA are epitopes of the EpCAM molecule that shows Lewis y glycosylation which epitopes are only present on aberrantly glycosylated EpCAM but not on normal EpCAM.

In particular the preferred TAA targets of the immunotherapeutic agent according to the invention are selected from the group of determinants derived from the group of antigens consisting of peptides or proteins, such as EpCAM, NCAM, CEA and T cell peptides, carbohydrates, such as aberrant glycosylation patterns, Lewis y, Sialyl-Tn, Globo H, and glycolipids, such as GD2, GD3 und GM2.

For active immunotherapy according to the invention a pharmaceutical preparation is formulated to include an immunogenic substance as described above that is preferably an immunogenic antibody or antibody derivative. This pharmaceutical preparation typically contains an amount of antibody or antibody derivative ranging between 0.01 μg and 10 mg. Depending on the nature of the antibody used as immunotherapeutic agent, the immunogenicity may be altered by xenogenic sequences or derivatization of the antibody. Besides, the use of adjuvants further increases the immunogenicity of the antibody. The immunogenic dose of a vaccine or an antibody or antibody derivative suitably formulated with an adjuvant is thus preferably ranging between 0.01 μg and 750 μg, more preferably between 100 μg and 1 mg, most preferably between 100 μg and 500 μg when used for active immunization. A vaccine designed for depot injection will however contain far higher amounts of the immunogenic substance, e.g. at least 1 mg up to 10 mg. The immunogen is thus delivered to stimulate the immune system over a longer period of time.

The immunogen used for active immunization according to the invention usually is provided as a ready-to-use pharmaceutical preparation in a single-use syringe containing a volume of 0.01 to 1 ml, preferably 0.1 to 0.75 ml. The vaccine solution or suspension thus provided is highly concentrated. The invention further relates to a kit for vaccinating patients, which comprises the vaccine and suitable application devices, such as a syringe, injection devices, pistols. etc.

The pharmaceutical preparation is particularly suitable for subcutaneous, intramuscular, intradermal or transdermal administration. Another possible route is the mucosal administration, either by nasal, peroral or rectal vaccination.

Further preferred is a pharmaceutical preparation comprising either the immunotherapeutic agent used as an immunogen for active immunotherapy or the immunotherapeutic agent for passive immunotherapy of the patients with rectal cancer or being at risk of rectal cancer, which preparation further comprises a pharmaceutically acceptable adjuvant and carrier to form a suppository for rectal administration.

Exemplary adjuvants improving the efficacy of the immunogen to produce an effective amount of the immunotherapeutic agent according to the invention are aluminium hydroxide (alum gel) or aluminium phosphate, growth factors, lymphokines, cytokines, like IL-2, IL-12, GM-CSF, interferons, or complement factors, e.g. C3d, liposomal preparations and formulations of additional antigens that are strong immunogens, such as tetanus toxoid, bacterial toxins, like pseudomonas exotoxins and derivatives of Lipid A.

The preferred vaccination regime of a pharmaceutical preparation used for active immunization according to the invention comprises an initial injection and preferably at least one booster injection. Booster injections are usually given in intervals between 2 and 40 weeks. A particular schedule is as follows: first injection on day 1 and further booster injections on days 15, 29 and 57 after the first vaccination. Preferably, further booster injections are 16, 24, 32 and 40 weeks after the first vaccination. Further vaccinations can be every 12 months, preferably every 6 months, more preferred every 3 months, most preferred every 2 months.

Determining seroconversion in the patient's serum proves the immune response received by application of a pharmaceutical preparation for active immunization according to the invention. Seroconversion is assayed by differential measurement of the binding of immunoglobulins of a patient's serum (before and after immunizations) to the antigen used for immunization. If the patient's serum does in fact show immunoglobulins specific against the antigen that had been applied, seroconversion has proven.

In case an immunotherapeutic agent is used for passive immunotherapy, the preferred amount of effective substance is between 1 mg and 1 g, preferably between 100 mg and 500 mg, depending on the maximal tolerated dose and the minimal effective dose as well as on the half life of the immunotherapeutic agent in the body. The pharmaceutical preparation suitable for passive immunotherapy is usually formulated together with appropriate carriers or buffers to obtain a preparation suitable for parenteral administration, preferably by the intravenous route.

For passive immunization, the preferred administration regimen of a pharmaceutical preparation containing the immunotherapeutic agent according to the invention is comprising several parenteral administrations in intervals of 1 week to 2 months, depending on the half life of the effective substance and the need of the patient. Typically infusions are given every 2 to 6 weeks for a period of several months up to one year per treatment course. As an example the patient receives first an infusion on day 1 and further infusions every four weeks. Preferably, the dose range of the first administration is between 250 mg and 1 g, for all further administrations the dose can be between 50 mg and 250 mg to keep the titre of the immunotherapeutic agent at a high level.

The pharmaceutical preparation used for either active or passive immunotherapy usually is storage stable at refrigerating temperature. However, preservatives, such as thimerosal or other agents of improved tolerability may be used to improve its storage stability to enable prolonged storage times even at elevated temperatures up to room temperature. The preparation according to the invention may also be provided in the frozen or lyophilized form, which is thawed or reconstituted on demand.

Preferred pharmaceutical formulations contain pharmaceutically acceptable carrier, such as buffer, salts, proteins or preservatives.

The immunotherapeutic treatment according to the invention can be employed in combination with conventional cancer therapies, such as surgery, chemotherapy and radiation therapy. Immunotherapy may be started for instance before or concomitant with standard chemotherapy, but also when chemotherapy has finished. The immunotherapeutic treatment according to the invention can also be done before or after surgical treatment, or even perioperatively in the course of a surgical intervention.

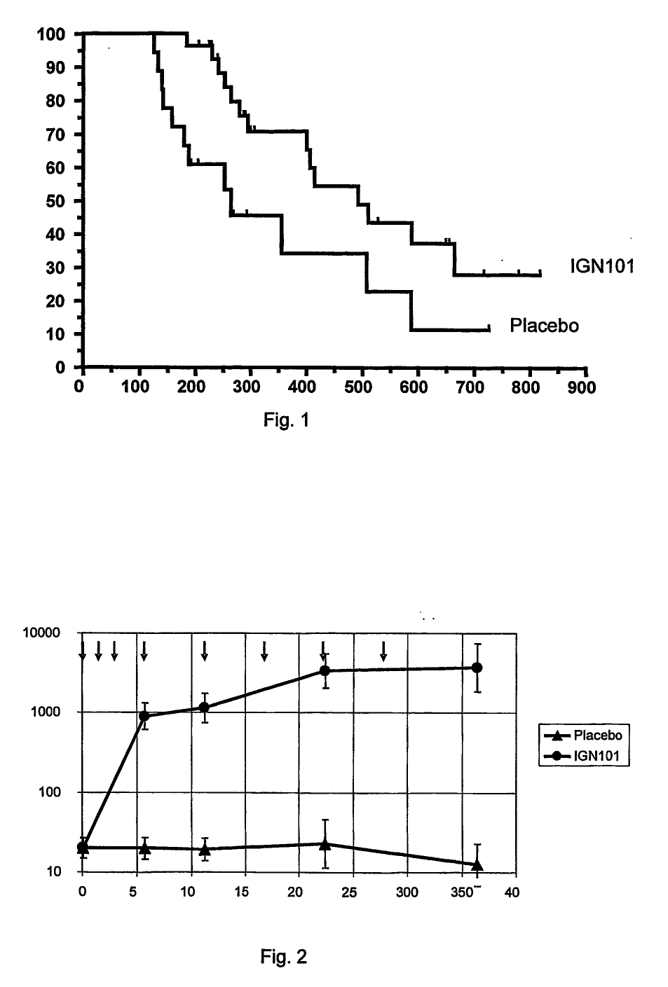

FIG. 1 shows the increase of survival rate of patients with stage IV rectal cancer with proven immune response. The survival was correlated with the immune response of the patients. The number of patients are 18 (placebo) and 28 (IGN101). On x-axis the days are applied, on y-axis the percent survival are applied.

FIG. 2 shows the geomean serum titres (95% conf. Intervals) of patients treated with placebo or mAb17-1A (IGN101). X-axis shows the number of days, y-axis the dilution.

The following example describes the invention in more detail, yet not limiting the scope of the invention.

Example: Clinical trial to evaluate safety, tolerability and immunogenicity of multiple doses of an EpCAM antibody.

The efficacy of multiple subcutaneous injections of the EpCAM antibody (mab17-1A, IGN101) formulated as a vaccine by adsorption onto Alum vs. placebo (adjuvant without the EpCAM antibody) was measured by determining overall survival in 25 patients with biopsy proven metastatic rectal cancer in stage IV.

All subjects received an initial course of 0.5 ml of the EpCAM antibody /placebo injected subcutaneously on day 1 (week 0), day 15 (week 2), day 29 (week 4) and day 57 (week 8), followed by further injections of the EpCAM antibody in weeks 16, 24, 32 and 40. A single vaccination dose consisted of 0.5 mg mab17-1A adsorbed on aluminum hydroxide as vaccine adjuvant in 0.5 ml physiological buffer.

The efficacy of mab17-1A was determined by the assessment of overall survival. Additionally, the time to occurrence of distant metastases (additional metastases for stage IV-patients) was assessed and tumor markers were measured.

The safety and tolerability of mab17-1A was determined by observing any adverse events related to study drug, serious adverse events related to study drug, and premature discontinuations related to study drug. Safety evaluations included clinical and laboratory assessments (physical examination, vital signs, hematology, serum chemistry, urinalysis, and adverse events).

Determining seroconversion in the patient's serum proves the immune response received by application of a pharmaceutical preparation for active immunization according to the invention. Seroconversion is assayed by differential measurement of the binding of immunoglobulins of a patient's serum (before and after immunizations) to the antigen used for immunization. For this test, seroconversion is defined by an at least five-fold increase of reactivity and a titre of 1:1000 of a patient's serum compared to the pre-immunization serum of the respective patient. A patient is considered seroconverted if seroconversion is achieved at two points after vaccination.

If the patient's serum does in fact show immunoglobulins specific against the antigen that had been applied, seroconversion has proven.

The immunogenicity of the EpCAM antibody was assessed by the total humoral immune response against mab17-1A (frequency of seroconversion) as follows.

The mouse monoclonal antibody used as vaccine antigen in mab17-1A was coated to ELISA microplate wells. Dilutions of the patient's serum are incubated in these wells. Binding of human immunoglobulin is detected by reaction of anti-human-immunoglobulinenzyme conjugate according to a common test protocol.

Survival of immune responders was analyzed by life-table methods. Comparison to non-responders (including the placebo group) was done by log-rank test. Time to transition to higher stage or addition of metastases was analyzed in the same way.

The results of the study according to the survival data are shown in FIG. 1. There were at least 4 immunizations/patient. Patients that did were treated with placebo did not show any immune response (n=28), and showed a lower survival rate compared to those patients who received at least 4 immunizations with the EpCAM antibody (n=18 patients). The median survival (days) are 264 for the placebo controlled patients, and 492 for mab17-1A treated patients, P=0.018 (log-rank). 1 year survival (%): 34.4 (placebo), 70.9 (mab17-1A treated), P=0.0067 (log-rank); 6 month survival (%): 66.7 (placebo controlled patients), 100 (mab17-1A treated patients), P=0.001 (log-rank)

FIG. 2 discloses the geomean serum titers (95% confidence intervals) of patients treated with placebo or mab17-1A.

1. A method for the immunotherapy of patients with rectal cancer or being at risk of rectal cancer which comprises administering to a patient an immunotherapeutic agent targeting a human tumor associated antigen (TAA) wherein the immunotherapeutic agent is an anti-body or derivative or fragment thereof having specificity for a tumor associated antigen in a dosage in the range of 0.1 to 1 mg.

2. The method according to claim 1 wherein the immunotherapeutic agent is an antibody of animal origin.

3. The method according to claim 1 or 2 wherein the immunotherapeutic agent is an antibody of hybridoma origin.

4. The method according to claim 1 or 2 wherein the immunotherapeutic agent is an antibody of recombinant origin.

5. The method according to claim 1 wherein the immunotherapeutic agent is a monoclonal antibody.

6. The method according to claim 1 wherein the immunotherapeutic agent is an idiotypic or anti-idiotypic antibody.

7. The method according to claim 1 wherein the immunotherapeutic agent is a protein or polypeptide or fragment of a TAA.

8. The method according to claim 7 wherein the immunotherapeutic agent is a protein or polypeptide or fragment of EpCAM.

9. The method according to claim 1 wherein the immunotherapy employs active immunization.

10. The method according to claim 1, wherein the tumor associated antigen is a cellular membrane antigen.

11. The method according to claim 1, wherein the tumor associated antigen is a cell adhesion protein.

12. The method according to claim 1, wherein the tumor associated antigen is selected from the group consisting of peptides, proteins, carbohydrates and glycolipids.

13. The method according to claim 1, wherein the tumor associated antigen is selected from the group consisting of CEA, EpCAM, N-CAM, TAG-72, MUC, Folate Binding Protein A-33, CA125, HER-2/neu, EGF-receptors, and MART.

14. The method according to claim 1, wherein the tumor associated antigen is selected from the group consisting of Lewis antigens, SialylTn, and GloboH.

15. The method according to claim 1, wherein the tumor associated antigen is selected from the group consisting of GD2, GD3 and GM3.

16. The method according to claim 1, wherein the immunotherapeutic agent is administered in a medicament suitable for administration by subcutaneous, intradermal, intramuscular injection, intravenous or by local or mucosal application.

17. The method according to claim 9, wherein the immunotherapeutic agent is administered by means of an initial injection and at least one further booster injection.

18. The method according to claim 17, wherein booster injections are given in intervals of 2-40 weeks, preferably at about 2, 4, 8, 16, 24, 32 and 40 weeks after the initial injection.

19. The method according to claim 18, wherein further injections are given 2 months, 3 months, 6 months and/or 12 months after the initial injection.

20. The method according to claim 17, wherein the injections are given every four weeks after an initial injection.

21. The method according to claim 1, wherein administration of said immunotherapeutic agent is combined with surgery, chemotherapy and/or radiation therapy.

22. The method according to claim 13, wherein said immunotherapeutic agent targets EPCAM.

23. The method according to claim 22 wherein the patients have rectal cancer stage III or stage IV.

24-29. (canceled)
